(19) United States
(12) Patent Application Publication
(10) Pub. No.: US 2015/0112722 A1
(43) Pub. Date: Apr. 23, 2015

(54) MEDICAL CONDITION TRACKING AND ANALYSIS

(71) Applicant: OSIA Medical, Inc., Sandy, UT (US)

(72) Inventors: Joshua Dees, Sandy, UT (US); Richard Hendershot, North Salt Lake, UT (US); Rob Reynolds, Kearns, UT (US)

(21) Appl. No.: 14/520,085

(22) Filed: Oct. 21, 2014

Related U.S. Application Data
(60) Provisional application No. 61/893,425, filed on Oct. 21, 2013.

(51) Int. Cl. A61B 5/00

(52) U.S. Cl. CPC A61B 5/7275; A61B 5/486; A61B 5/7465; A61B 5/0022
USPC 705/3

(57) ABSTRACT

An apparatus, system, and method are disclosed for medical tracking and analysis. A data module receives subjective and objective data associated with a medical condition for a patient. A trend module determines a trend for the medical condition associated with the patient over a period of time based on the subjective and objective data. A factor module identifies one or more factors that affect the medical condition and calculates one or more weighted values for the one or more factors based on the trend.

### CROSS-REFERENCES TO RELATED APPLICATIONS

This application claims the benefit of U.S. Provisional Patent Application No. 61/893,425 entitled “MEDICAL CONDITION TRACKING AND ANALYTICS BASED ON SUBJECTIVE AND OBJECTIVE DATA” and filed on Oct. 21, 2013, for Joshua Dees, which is incorporated herein by reference.

### FIELD

This invention relates to medical treatment and more particularly relates to tracking and analyzing data related to a medical condition.

### BACKGROUND

It is common for people to suffer from chronic medical conditions, such as asthma, allergies, diabetes, hypertension, etc. Chronic conditions adversely affect the lives of the people who have the conditions, and place a great burden on the medical industry. In many cases, patients and physicians consistently work to keep chronic conditions under control, which consumes the time of both the patient and the physician, and which can be very costly in terms of medication costs, medical care costs, cost due to lost work, etc. In some cases, when a chronic condition is not well controlled, the condition can lead to serious illness/injury or even death.

Optimizing treatment routines for chronic conditions has received a great deal of attention. For example, in the context of asthma, physicians have developed control tests, which are used to gauge how well a condition is being kept in control. A control test may ask a patient a series of questions (e.g., gauging how much the condition has interfered with work or personal life, whether the condition is interfering with sleep, how often medication is being used, etc.) and assign a score to the patient based on his answers. These scores can then be used to determine whether the patient should alter his medication routine, whether the patient should schedule an appointment with his physician, etc.

Despite the advances that have been made in the treatment of chronic conditions (e.g., in terms of medications, control tests, etc.), chronic conditions very often remain difficult to keep under control.

### SUMMARY

An apparatus for medical condition tracking and analysis is disclosed. A system and method also perform the functions of the apparatus.

In one embodiment, an apparatus for medical condition tracking and analysis includes a data module that receives subjective and objective data associated with a medical condition for a patient. In a further embodiment, the apparatus includes a trend module that determines a trend for the medical condition associated with the patient over a period of time based on the subjective and objective data. In some embodiments, the apparatus includes a factor module that identifies one or more factors that affect the medical condition. In one implementation, one or more weighted values are calculated for the one or more factors based on the trend. In certain embodiments, at least a portion of the data module, the trend module, and the factor module comprise one or more of hardware and executable code stored on one or more computer readable storage media.

In certain implementations, the trend module updates the trend for the medical condition associated with the patient in real-time in response to receiving subjective and/or objective data. In another embodiment, the factor module determines the one or more weighted values by correlating one or more data points of the subjective data with one or more data points of the objective data. In a further implementation, the apparatus includes a recommendation module that generates one or more recommendations related to the medical condition based on the weighted values of the one or more factors. In some implementations, the apparatus includes a prediction module that forecasts a state of the medical condition at a future point in time based on the one or more factors and forecasted objective data.

In one embodiment, the apparatus includes a report module that generates one or more reports for the patient based on a state of the patient's medical condition. In some implementations, the one or more reports is accessible to a client device associated with the patient's doctor. The apparatus, in another embodiment, includes a messaging module that generates one or more messages for a patient. In one embodiment, the one or more messages are sent in response to input received from the patient's doctor. In certain implementations, the factor module identifies the one or more factors based on one or more outlier data points of the determined trend. In one embodiment, the outlier data points indicate one or more abnormalities associated with a state of the patient's medical condition.

In a further embodiment, the subjective data comprises qualitative feedback provided by the patient and related to the medical condition, the patient feedback being received from a client device associated with the patient. The subjective data, in another embodiment, comprises event data received from a client device associated with the patient. In certain implementations, the event data comprises a self-reported measurement of the medical condition associated with the patient at a point in time.

In certain embodiments, the objective data comprises environmental data. In various implementations, the environmental data is received from one or more remote servers storing the environmental data. In yet another embodiment, the environmental data comprises one or more of weather data, air quality data, pollen data, pollution data, ozone data, pressure data, and particulate matter data. In one implementation, the objective data comprises data reported by the patient, which comprises one or more quantifiable factors associated with the medical condition.

A system for medical condition tracking and analysis includes, in one embodiment, a client device associated with a patient and a client device associated with a doctor for the patient. In certain embodiments, the system also includes a remote server communicatively coupled to the client device associated with a patient and the client device associated with the doctor for the patient.

In some embodiments, the system includes a data module that receives subjective and objective data associated with a medical condition for a patient. In a further embodiment, the system includes a trend module that determines a trend for the medical condition associated with the patient over a period of time based on the subjective and objective data. In some embodiments, the system includes a factor module that identifies one or more factors that affect the medical condition. In one implementation, one or more weighted values are calculated for the one or more factors based on the trend. In certain embodiments, at least a portion of the data module, the trend module, and the factor module comprise one or more of hardware and executable code stored on one or more computer readable storage media.

In certain implementations, the trend module performs one or more statistical analyses on the subjective and objective data on the remote server. In some embodiments, the trend is generated as a function of one or more of the statistical analyses. In a further embodiment, the system includes a report module that sends the results of the one or more statistical analyses to the client device associated with the doctor of the patient.

In yet another embodiment, the report module presents the results of the one or more statistical analyses on a graphical interface for the client device associated with the doctor of the patient. In some implementations, the objective data is received from one or more external data sources comprising one or more remote servers storing environmental data. In some embodiments, the environmental data is associated with the patient's medical condition.

A method for medical condition tracking and analysis, in one embodiment, includes receiving, by a processor, subjective and objective data associated with a medical condition for a patient. In a further embodiment, the method includes determining a trend for the medical condition associated with the patient over a period of time based on the subjective and objective data. In some implementations, the method includes identifying one or more factors that affect the medical condition, wherein one or more weighted values are calculated for the one or more factors based on the trend. In one embodiment, the method further includes forecasting a state of the medical condition at a future point in time based on the one or more factors and forecasted objective data.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a schematic block diagram illustrating one embodiment of a system for medical condition tracking and analysis;

FIG. 2 is a schematic block diagram illustrating one embodiment of a module for medical condition tracking and analysis;

FIG. 3 is a schematic block diagram illustrating one embodiment of another module for medical condition tracking and analysis;

FIG. 4 is a schematic block diagram illustrating one embodiment of another system for medical condition tracking and analysis;

FIG. 5 is a diagram of a trend line for medical condition tracking and analysis;

FIG. 6 is a schematic flow chart diagram illustrating one embodiment of a method for medical condition tracking and analysis; and

FIG. 7 is a schematic flow chart diagram illustrating one embodiment of a method for medical condition tracking and analysis.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

These features and advantages of the embodiments will become more fully apparent from the following description and appended claims, or may be learned by the practice of embodiments as set forth hereinafter. As will be appreciated by one skilled in the art, aspects of the present invention may be embodied as a system, method, and/or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” or “system.” Furthermore, aspects of the present invention may take the form of a computer program product embodied in one or more computer readable medium(s) having program code embodied thereon.

Many of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of program code may, for instance, comprise one or more physical or logical blocks of computer instructions which may, for instance, be organized as an object, procedure, or function. Nevertheless, the executables of an identified module need not be physically located together, but may comprise disparate instructions stored in different locations which, when joined logically together, comprise the module and achieve the stated purpose for the module.

Indeed, a module of program code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network. Where a module or portions of a module are implemented in software, the program code may be stored and/or propagated on in one or more computer readable medium(s).

The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (“RAM”), a read-only memory (“ROM”), an erasable programmable read-only memory (“EPROM” or Flash memory), a static random access memory (“SRAM”), a portable compact disc read-only memory (“CD-ROM”), a digital versatile disk (“DVD”), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The schematic flowchart diagrams and/or schematic block diagrams in the Figures illustrate the architecture, functionality, and operation of possible implementations of apparatuses, systems, methods and computer program products according to various embodiments of the present invention. In this regard, each block in the schematic flowchart diagrams and/or schematic block diagrams may represent a module, segment, or portion of code, which comprises one or more executable instructions of the program code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and program code.

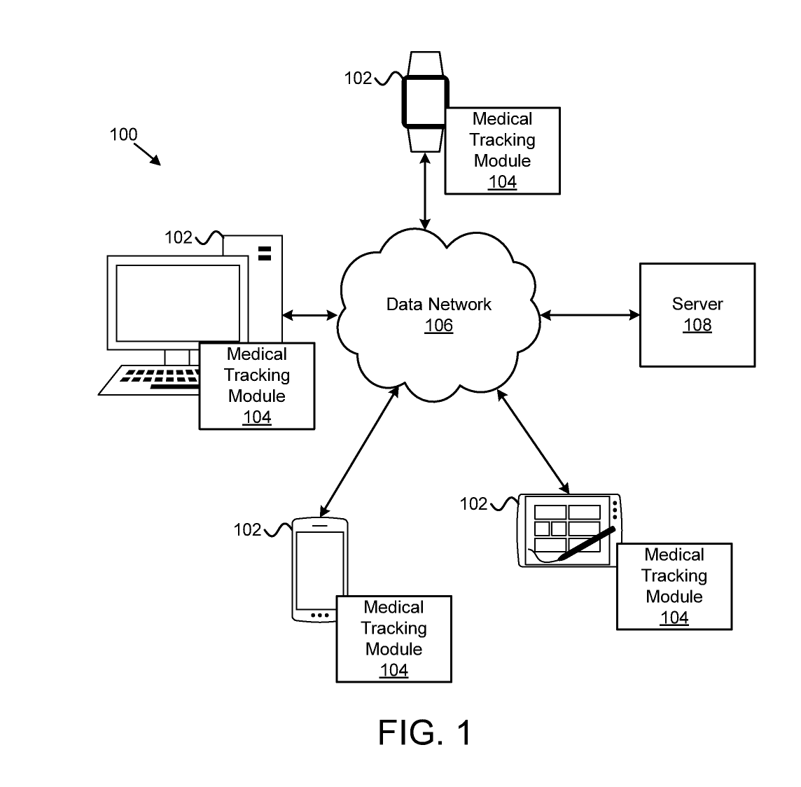

FIG. 1 depicts one embodiment of a system 100 for medical condition tracking and analysis. In one embodiment, the system 100 includes information handling devices 102, medical tracking modules 104, data networks 106, and servers 108. Even though a specific number of information handling devices 102, medical tracking modules 104, data networks 106, and servers 108 are depicted in FIG. 1, one of skill in the art will recognize that any number of information handling devices 102, medical tracking modules 104, data networks 106, and servers 108 may be included in the system 100.

In one embodiment, the information handling devices 102 comprise computing devices, such as desktop computers, laptop computers, personal digital assistants (PDAs), tablet computers, smart phones, smart televisions (e.g., televisions connected to the Internet), or the like. In some embodiments, the information handling devices 102 comprise wearable devices, such as smart watches, fitness bands, optical head-mounted displays, or the like. In one embodiment, the information handling devices 102 include one or more cameras and sensors, such as heart rate monitors, pressure sensors, location sensors, and/or the like.

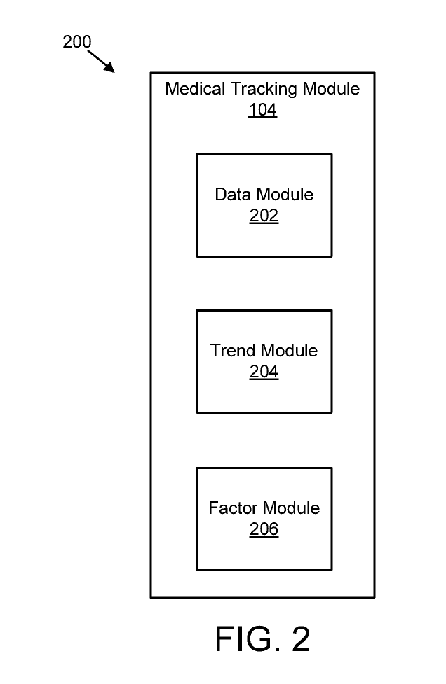

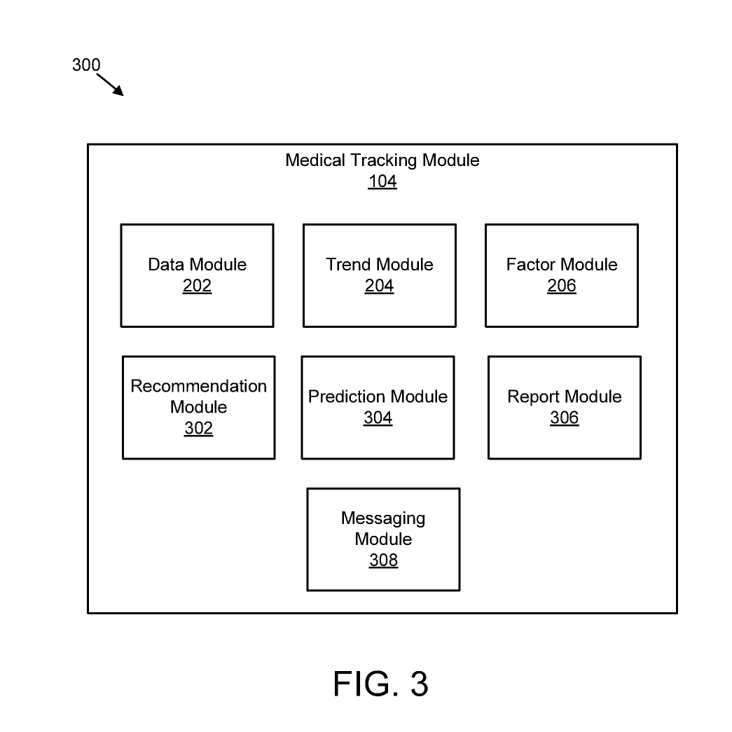

The information handling devices 102, and/or the server 108, may include an embodiment of the medical tracking module 104. In certain embodiments, the medical tracking module 104 is configured to receive subjective and/or objective data from at least one client device that is associated with a patient. In one embodiment, the medical tracking module 104 is configured to also receive objective data from one or more external, or third-party maintained, data sources. In some embodiments, the medical tracking module 104 is configured to determine a trend for a medical condition associated with the patient over a period of time based on the subjective and the objective data. In a further embodiment, the medical tracking module 104 is configured to identify one or more factors that affect the medical condition of the patient and calculate weighted values for the one or more factors based on the determined trend. In this manner, the medical tracking module 104 may provide, real-time, up-to-date, tracking of a user's medical condition and generate recommendations and forecasts based on the trend data and the determined factors. In certain embodiments, the medical tracking module 104 includes various modules that perform one or more of the operations of the medical tracking module 104, which are described in more detail below with reference to FIGS. 2 and 3.

The data network 106, in one embodiment, comprises a digital communication network that transmits digital communications related to tracking a patient's medical condition. The data network 106 may include a wireless network, such as a wireless cellular network, a local wireless network, such as a Wi-Fi network, a Bluetooth® network, a near-field communication (NFC) network, an ad hoc network, and/or the like. The data network 106 may include a wide area network (WAN), a storage area network (SAN), a local area network (LAN), an optical fiber network, the internet, or other digital communication network. The data network 106 may include two or more networks. The data network 106 may include one or more servers, routers, switches, and/or other networking equipment. The data network 106 may also include computer readable storage media, such as a hard disk drive, an optical drive, non-volatile memory, random access memory (RAM), or the like.

In one embodiment, the servers 108 include computing devices, such as desktop computers, laptop computers, mainframe computers, cloud servers, virtual servers, and/or the like. In some embodiments, the servers 108 are configured as application servers, email servers, database servers, file servers, game servers, home servers, media servers, web servers, and/or the like. In certain embodiments, the servers 108 are configured to store the subjective and/or objective data received by the medical tracking module 104. In some embodiments, the servers 108 are also configured to analyze the subjective and/or objective data, e.g., by running various statistical analyses on the data. In one embodiment, the servers 108, including the data on the servers 108, are configured to be accessed by one or more information handling devices 102 through the network 106. The servers 108 may include at least a portion of the medical tracking module 104 and perform one or more operations of the medical tracking module 104.

FIG. 2 depicts one embodiment of a module 200 for medical condition tracking and analysis. In one embodiment, the module 200 includes an embodiment of a medical tracking module 104. The medical tracking module 104, in certain embodiments, includes various embodiments of a data module 202, a trend module 204, and a factor module 206, which are described in more detail below.

The data module 202, in one embodiment, is configured to receive subjective data from a client device associated with a patient. As used herein, subjective data comprises information reported by a patient that is related to a patient's medical condition and may be used to determine how a patient feels at a given moment in time and/or how the patient's medical condition is being maintained. In some embodiments, the data module 202 receives subjective data in the form of responses to questionnaires, surveys, polls, quizzes, assessments, or the like that present specific questions related to the medical condition that is being tracked. In certain embodiments, this type of user-provided data is known as control data. Thus, by way of example, the data module 202 may receive responses to an asthma control evaluation mechanism, which may be used to gauge how the patient feels at a specific point in time, including how often the condition is affecting the patient, the extent of the particular symptoms, or the like. Examples of asthma control evaluation mechanisms may include the Asthma Control Test (ACT) and the Pediatric Asthma Control and Communication Instrument (PACCI). In some embodiments, the control test data may focus on periods of time, such as the last several days, the prior week, the time since the last control test, or the like.

In a further embodiment, the data module 202 may receive subjective data that includes event logging data, such as how well the patient feels (e.g., poor, moderate, well), the extent to which the patient feels the condition is affecting him/her, or the like. In some embodiments, event logging data focuses on a specific point in time, e.g., how the patient feels at the moment in time that the event log was entered. For example, the data module 202 may receive event logging data for a patient that suffers an asthma attack in response to the patient entering the event logging data after the asthma attack occurred, e.g., the patient may enter how severe they felt the asthma attack was, how long it lasted, where they were when it occurred, or the like. In this manner, the medical tracking module 104, using the event logging data together with the objective data described below, may determine what factors may affect the patient's medical condition and may provide recommendations and/or guidance to help maintain or control the patient's condition.

In one embodiment, the data module 202 receives objective data reported by a patient on the device 102 associated with the patient. User-provided objective data received by the data module 202 may also be known as “interactive” objective data. For example, the data module 202 may receive an inhaler usage count, a frequency or number of exposures to allergens (e.g., plants, pets, etc.) for a time period, a number of times a day a medication is taken, and/or the like, as reported by the patient. The data module 202 may present a questionnaire, survey, or the like to the user that comprises questions designed to elicit the “interactive” objective data. Thus, unlike the subjective data, which may substantially comprise qualitative data, the objective data may substantially comprise quantifiable data.

In one embodiment, the data module 202 receives objective data from one or more external sources. Objective data, as user herein, may include any data that can be obtained objectively and that is relevant to a medical condition of the patient. For example, in the case of an asthma or allergy medical condition, the objective data may comprise data for various environmental factors, including, but not limited to, weather conditions (e.g., humidity, temperature, cloud cover, precipitation type or extent, wind direction/magnitude, biometric pressure, or the like), pollen levels, types of pollen, ozone, particulates (e.g., PM10, PM2.5, or the like), air quality, pollution levels, and/or the like. Depending on the medical condition, other objective data may include physiological data (e.g., blood pressure, heart rate, blood glucose levels, or the like), medication data (e.g., a type of medication being used, a history of medication use, frequency of medication use, or the like), medical history data for the patient, infectious disease data, and/or the like.

In one embodiment, the data module 202 receives objective data from one or more external sources that comprise one or more third-party data servers. For example, the data module 202 may receive air quality data from a data store associated with, or maintained by, the Environmental Protection Agency (EPA). The data module 202 may be communicatively coupled to various data stores that comprise environmental data or other objective data that is maintained by a third-party agency, such as the Centers for Disease Control (CDC), the National Oceanic and Atmospheric Administration (NOAA), or the like. Other data sources may include pollen-related databases, which may comprise pollen data collected from one or more pollen collection stations. The pollen collection stations, for example, may be communicatively coupled via a data network 106 such that pollen data may be collected for a plurality of different locations. Additionally, the data module 202 may receive data from various personal environmental sensors associated with the patient, such as indoor air quality sensors, barometric sensors, temperature sensors, and/or the like. The personal environmental sensors may be associated with one or more information handling devices 102, e.g., the sensors may be integrated with or otherwise connected to a patient's smart phone, tablet computer, or the like, which may allow other information handling devices 102 to access collected environmental data via an application programming interface (API).

In certain embodiments, the data module 202 receives objective data from the client device associated with the patient. For example, after a patient provides subjective feedback for a medical event, the data module 202 may receive a timestamp, a location, or the like, from the patient's smart phone, fitness band, smart watch, or the like. In some embodiments, the data module 202 receives physiological data from one or more sensors of the patient's device, such as the patient's heart rate as measured by a heart rate sensor, the patient's oxygen levels as measured by an oxygen level sensor, and/or the like. For example, a diabetic patient may have his glucose or insulin levels measured using a sensor of a device 102 configured to measure glucose and/or insulin levels.

The trend module 204, in one embodiment, is configured to determine a trend for a medical condition associated with the patient over a period of time based on the subjective and the objective data. In some embodiments, the trend module 204 tracks subjective data received by the data module 202 from a client device associated with the patient for a specific period of time to determine how a patient's medical condition is trending. For example, based on historical trend data and current subjective and/or objective data, the trend module 204 may determine that the patient's medical condition is trending upwards (meaning in a controlled or well-maintained direction) or downward (meaning in an uncontrolled or not-well-maintained direction). In one embodiment, the trend module 204 determines a wellness score, rating, rank, or the like, based on the subjective data received by the data module 202. For example, a wellness score may be determined based on a scoring scale associated with a control test, e.g., a questionnaire, a poll, a quiz, a survey, or the like. In such an embodiment, a patient may specify how he is feeling at a particular time, via either medical event data or control test data. Based on the received subjective data, the trend module 204 may determine an overall wellness score, which represents a wellness status for the patient, based on the subjective data and determine whether the patient's medical condition is trending high (meaning that the medical condition is being controlled correctly) or low (meaning that the medical condition is not being controlled correctly).

In certain embodiments, the trend module 204 “enriches” the subjective data based on the objective data received by the data module 202. The trend module 204 may “enrich” the data by incorporating one or more objective data points into the subjective data provided by the patient. For example, the trend module 204 may incorporate air quality data, pollen data, or the like, for a particular time period into subjective data provided by an asthmatic patient. In this manner, the trend module 204 may determine an accurate score or rating for the patient's medical condition at a specific point in time in order to determine how the patient's medical condition is trending in relation to previously recorded data points and current objective data values. As used herein, “enriching” the subjective data may include performing one or more mathematical operations, such as regression analyses, correlations, or the like, on the subjective and the objective data in order to determine an overall wellness score, rating, rank, or the like that represents a wellness status for the patient.

In one embodiment, the trend module 204 may determine a plurality of wellness scores that collectively, or independently, represent a wellness status for the patient. For example, the trend module 204 may determine an evaluation score that represents the long-term trend for the patient. The evaluation score may be determined by a predetermined scoring system for the evaluation method being used, such as a predetermined or predefined scoring system for the PACCI or the ACT. The trend module 204, in one embodiment, normalizes the evaluation score across a predetermined domain, such as a domain range from 0 to 100, based on samples predetermined by the evaluation method being used, e.g., an evaluation method may specify that the evaluation be administered to the patient every day, every other day, or the like.

In another example, the trend module 204 may determine an event score that represents the substantially real-time status of the patient's medical condition. In such an embodiment, the trend module 204 samples the substantially real-time status of the patient's medical condition as often as the patient reports how they are feeling. The trend module 204 may determine a score for the patient's wellness based on the patient's feedback. For example, the trend module 204 may present an interactive interface to a patient that comprises a “slider” interface element that allows a patient to specify how they are feeling on a scale of 0 to 100, with 0 being poor and 100 being good, by sliding the slider towards one of the two ends of the scale. Because the patient's feedback is sampled in real-time, or substantially in real-time, the trend module 204 may “enrich” the patient's feedback, e.g., the subjective data, with objective (e.g., “interactive” objective and/or environmental objective) data to create a more complete picture of the patient's wellness status, including determining the possible effects that the patient's environment is having on them at that moment.

In some embodiments, the trend module 204 tracks both the evaluation score and the event score, in conjunction with the “enriched” data, in order to provide an overall wellness status of the medical condition. In one embodiment, the factor module 206, described below, may use the wellness status to determine one or more factors that may affect the patient's medical condition. In some embodiments, the report module 306, also described below, may report the patient's wellness status to the patient's doctor via an interface, electronic message, or the like, who may use the wellness status to determine a course of action for the patient.

In one embodiment, the trend module 204 presents the patient's trend data on an interactive interface. In one embodiment, the trend module 204 presents an interface similar to a line graph that includes a trend line, with the time period being measured along the x-axis and the scores/ratings/rankings of the trend data being measured along the y-axis. In such an embodiment, a patient may select, click on, hover over, or the like, different data points of the trend line to view additional information about the data point, such as various objective data values, subjective data values, or the like.

In certain embodiments, the trend module 204 presents a multi-dimensional trend such that various time periods, demographic factors, objective data types, or the like may be represented by the trend. For example, the trend module 204 may present trend data for a specific time period (day, month, year), for a season (spring, summer, fall, winter), for different genders or ages, or the like. In certain embodiments, the trend module 204 updates the trend data in real-time in response to receiving subjective and/or objective data. For example, a patient may respond to a medical questionnaire in the morning, which may cause the trend module 204 to update the trend data in at that time. Subsequently, the patient may also log event data in the evening, and the trend module 204 may update the trend data in response to receiving the event data.

In one embodiment, the factor module 206 is configured to identify one or more factors that affect the patient's medical condition. As used herein, a factor that affects the patient's medical condition may comprise environmental, genetic, hereditary, demographic, or the like factors that may have no effect, a positive effect (an alleviating effect), or a negative effect (an exacerbating effect) on the patient's medical condition. In some embodiments, the factor module 206 determines one or more factors based on trend data as determined by the trend module 204. For example, the factor module 206 may analyze various data points of the trend data to determine one or more factors that may affect how the patient was feeling at a certain point.

In one embodiment, the factor module 206, in order to determine one or more factors that affect the patient's medical condition, correlates one or more data points based on the subjective data with one or more data points based on the objective data. For example, the factor module 206 may correlate a patient's response that he suffered an asthma attack in a certain location with pollen levels, air quality, or the like in that location. The patient, at a later point in time, may report an asthma attack at a different location, and the factor module 206 may correlate the patient's responses related to the asthma attack with the pollen levels, air quality, or the like at the different location. Over time, the factor module 206, based on an analysis of a plurality of data points derived from the trend data, may determine that certain pollen levels or pollen types have a negative effect on the patient's asthma condition.

In certain embodiments, the factor module 206, after determining a plurality of factors that may affect the patient's medical condition, assigns a weighted value to each of the factors. In some embodiments, the factor module 206 assigns a higher weighted value to factors that have a greater effect on the patient's medical condition than other factors. For example, the factor module 206 may assign a higher weight to pollen factors than to barometric pressure factors for a patient that suffers from seasonal allergies. Based on the determined factors and the accompanying weights, a doctor or a patient can quickly determine which factors exacerbate or alleviate the medical condition.

The weighted values determined by the factor module 206 may be based on an occurrence of a factor within one or more data points of the trend data. As such, the factor module 206 may calculate one or more statistical analyses, such as regressions and correlations, using one or more of the subjective data points and the objective data points. For example, if a patient that suffers from seasonal allergies reports suffering from itchy eyes and a runny nose at the same location every day, such as a home or office, the factor module 206 may run various correlations based on the patient's location and one or more objective factors to determine which environmental factors may contribute to the patient's poor medical condition at that time.

In some embodiments, the factor module 206 uses one or more outlier data points in the trend data in order to determine one or more factors that affect the patient's medical condition. Outlier data points, as used herein, may comprise abnormal or unexpected data points in the trend data. However, outlier data points may also indicate new or unknown factors that contribute to the patient's medical condition. For example, a patient's trend data may indicate that he has kept his asthma condition well-maintained or controlled for the past year. However, if the patient recently moved to a new location or started work at a new job site, his medical condition may start to worsen or become less maintained, which may be indicated by an unexpected change in the trend data.

In response to the unexpected or abnormal trend data, the factor module 206 may analyze the outlier data to determine which factors may have caused the change in the patient's maintenance of his medical condition. Thus, for example, if an asthma patient recently moved to a new home that has different pollen types and levels than his old home, and the patient reports suffering from more asthma attacks than usual, the factor module 206 may determine which of the pollen types has an effect on the patient's asthma condition in response to detecting a decline in the patient's medical condition trend.

In certain embodiments, the factor module 206 may implement business intelligence methods to determine a relationship between the subjective data and the objective, and to assign weights to the determined relationships. For example, as described above, the business intelligence analytics may compare, correlate, or otherwise calculate values describing relationships between subjective data and environmental factors, demographic factors, or the like. In certain embodiments, when the patient's medical condition is under control, the values describing the relationships between subjective data and the objective data may me be low, which may indicate that the relationship is not very strong. By contrast, when the medical condition is not being maintained properly, the correlations between the subjective data and the objective data will be higher, indicating that there may be a stronger relationship between certain factors, which the factor module 206 may assign high weights to.

In some embodiments, the factor module 206 determines one or more factors that may have an effect on the medical condition based on cross-analysis of the patient's subjective data with other patients' data who suffer from the same or similar conditions. For example, the factor module 206 may determine which factors affect treatment of the medical condition by monitoring, tracking, or the like, the treatment of members of a community having similar medical conditions, as a whole, as the objective data (e.g., environmental conditions) changes. As such, the factor module 206 may determine factors to the extent to which individual objective data points (e.g., environmental conditions such as pollen, particulate matter, ozone, etc.) affect people having similar medical conditions. In addition, the factor module 206 may determine how the severity of medical conditions in a region change over time as environmental conditions change. For example, the factor module 206 may determine the prevalence of asthma attacks in a region (e.g., city, county, state, etc.) over a period of time (e.g., months, years, decades, etc.) as air quality improves/degrades.

FIG. 3 depicts one embodiment of a module 300 for medical condition tracking and analysis. In one embodiment, the module 300 includes an embodiment of a medical tracking module 104. The medical tracking module 104, in certain embodiments, includes a data module 202, a trend module 204, and a factor module 206, which may be substantially similar to the data module 202, the trend module 204, and the factor module 206 described above with reference to FIG. 2. In a further embodiment, the medical tracking module 104 includes a recommendation module 302, a prediction module 304, a report module 306, and a messaging module 308.

In one embodiment, the recommendation module 302 is configured to generate one or more recommendations related to treatment of the medical condition of the patient. In certain embodiments, the recommendation module 302 bases its recommendations on the weighted values of the one or more factors. For example, the recommendation module 302 may generate recommendations for an asthma patient that suggest types of pollen to avoid, the times when pollen levels are at their lowest/highest, or the like, based on the weights assigned to pollen factors by the factor module 206.

In some embodiments, the recommendation module 302 sends the one or more recommendations to the patient via the client device for the patient. In a further embodiment, the recommendation module 302 sends the one or more recommendations to the patient's doctor via the client device for the doctor. In this manner, the patient and the doctor may work together to help maintain or control the patient's medical condition in order to increase the effectiveness of the doctor's treatment of the patient.

The recommendation module 302, in certain embodiments, determines treatment recommendations, medicine recommendations, office visit recommendations, follow-up recommendations, or the like, as they are related to the patient's medical condition. For example, the recommendation module 302 may recommend to a doctor that the doctor see a particular asthma patient once a week. Additionally, the recommendation module 302 may recommend that the patient take a particular asthma medicine for a recommended period of time, such as two days, two weeks, or the like. As described below, the messaging module 308 may send the one or more recommendations to a device 102 associated with a patient from a device 102 associated with the patient's doctor.

In certain embodiments, the recommendation module 302 determines one or more recommendations or suggestions to help a doctor maintain her workload of patients. For example, the recommendation module 302 may recommend that a doctor set up an appointment with three of the ten patients that the doctor regularly sees because the three recommended patients' medical conditions may be trending downward, indicating a change in the control of the patients' medical condition.

The prediction module 304, in one embodiment, is configured to forecast a state of the medical condition at a future point in time based on the one or more factors and forecasted objective data. As used herein, forecasted objective data may comprise an estimate or a forecast of objective data at some point in the future. For example, the prediction module 304 may forecast air quality readings for a week, a month, or the like from a current point in time. In certain embodiments, the prediction module 304 forecasts objective data values based on historical objective data points, trends of historical objective data, or the like.

The prediction module 304 may determine a state of the patient's medical condition at a future point in time based on one or more factors determined by the factor module 206 that have been determined to have an effect on the patient. For example, the prediction module 304 may forecast that a patient may suffer an allergic reaction to a particular pollen that typically displays high pollen levels in two weeks. The prediction module 304 may implement one or more analytical or business intelligence methods, as described above to determine whether a forecasted objective data set may have an effect on a patient. In this manner, the doctor and the patient can take proactive, preventative measures to help control the patient's medical condition in anticipation of the increased pollen levels.

In a similar manner, the patient's doctor may coordinate his workload, inventory, time, or the like, based on the predictions determined by the prediction module 304. For example, if the prediction module 304 forecasts an increase in pollen levels that may affect one or more of his patients, the doctor can take proactive steps to arrange office visits with his effected patients, order supplies of medicines that may be needed to treat the patients' medical conditions, or the like.

The report module 306, in certain embodiments, is configured to generate one or more reports for the patient based on a state of the patient's medical condition. For example, as described above, the report module 306 may generate a graph or chart of the trend of the patient's medical condition over a predetermined period of time. The report module 306, in a further embodiment, may generate a summary report, a forecast report, a historical report, or the like associated with a patient's medical and treatment history. The report module 306 may send generated reports to the client devices associated with a patient and/or a doctor.

The report module 306 may also generate reports for the patients in a doctor's patient pool. In some embodiments, the report module 306 generates a report that provides a summary of the states of the medical conditions of all the doctor's patients. The report module 306 may, for example, generate a report showing a percentage of the doctor's patients that report feeling good, moderate, or bad. Similarly, the report module 306 may generate a report listing the doctor's patients that may see a change in their medical condition due to upcoming, forecasted changes in environmental data. For example, if the pollen levels for a particular type of pollen is forecasted to increase within the next few weeks, the report module 306 may generate a report listing the patients who have had a history of being affected by higher levels of the particular pollen (which may be based on the weighted values for the pollen as determined by the factor module 206). In this manner, the doctor may better prepare, plan, organize, or the like the treatment that he provides to his patients.

Similarly, the report module 306 may send a patient reports regarding the status of a patient's medical condition, including historical trends and predictions about the factors or conditions that may affect the patient's medical condition. For example, the report module 306 may generate a report stating that provides a graph of the patient's trending medical condition as reported over the past three months, one or more recommendations personally tailored to the patient by the recommendation module 302 (e.g., recommendations may include when to schedule an office visit, when to refill a prescription, or the like), one or more predictions related to the patient's medical condition (e.g., changing seasons may cause increased or decreased pollen levels that may affect an allergy patient's medical condition), or the like.

In one embodiment, the report module 306 presents one or more reports on an interface associated with the client devices of the patient and/or doctor. For example, the report module 306, on the client device associated with the doctor, may present one or more widgets that show a list of the doctor's patients and their current medical statuses, a chart showing which patients are trending up or down, a messaging gateway, described below, that allows the doctor to send messages to patients, or the like. In some embodiments, the report module 306 provides a similar interface to a patient that includes information specific to the patient, such as the patient's trending medical history, a history of the patient's self-reported diagnoses, factors that have been determined (e.g., by the factor module 206) to affect the patient's medical condition, or the like.

In this manner, the patient's doctor may increase the efficiency and effectiveness of his medical team. By providing insight in to the status and prioritization of the client load, for example, based on key performance indicators (KPIs) or other factors, the efforts of the medical team can be focused on those who need it most. For example, the medical team may be notified when control test data shows that the effectiveness of the control for certain patients' conditions has been degrading, so that the medical team may proactively schedule appointments with those patients. In some embodiments, the report module 306 may provide to the medical team specific reports for upcoming appointments, which may contain analytics suggesting possible causes for loss of control in the patient's medical condition. In yet another example, the physician may be provided with a report showing the level of participation of patients within the system, e.g. whether patients are submitting the minimum required control tests and events. For those patients who do not meet the minimum, the medical team can prompt them to participate as required.

In one embodiment, the messaging module 308 is configured to generate, send, and/or receive one or more messages associated with the patient's medical condition. The messaging module 308, for example, may be configured to automatically send a message to a patient to remind the patient to report how they are feeling in response to the patient failing to report their current medical condition (e.g., failing to provide subjective data for their medical condition) for the last three days. Similarly, the messaging module 308 may send a proactive message to a patient to remind them to take their medication, to report the current state of their medical condition, to warn them of upcoming environmental conditions that may affect their medical condition, or the like. In some embodiments, the messaging module 308 sends messages according to a predetermined schedule.

For example, a doctor may set messages to be sent to patients a day before their scheduled office visit, a few hours after an office visit, three days after the patient fails to report how they feel, or the like. In certain embodiments, the messaging module 308 sends a message to a doctor or a patient in response to user input. For example, a doctor may send a custom message to a patient. Similarly, a patient may send a message to a doctor or one or more other patients within the doctor's pool of patients or a similar community of patients that have similar medical conditions.

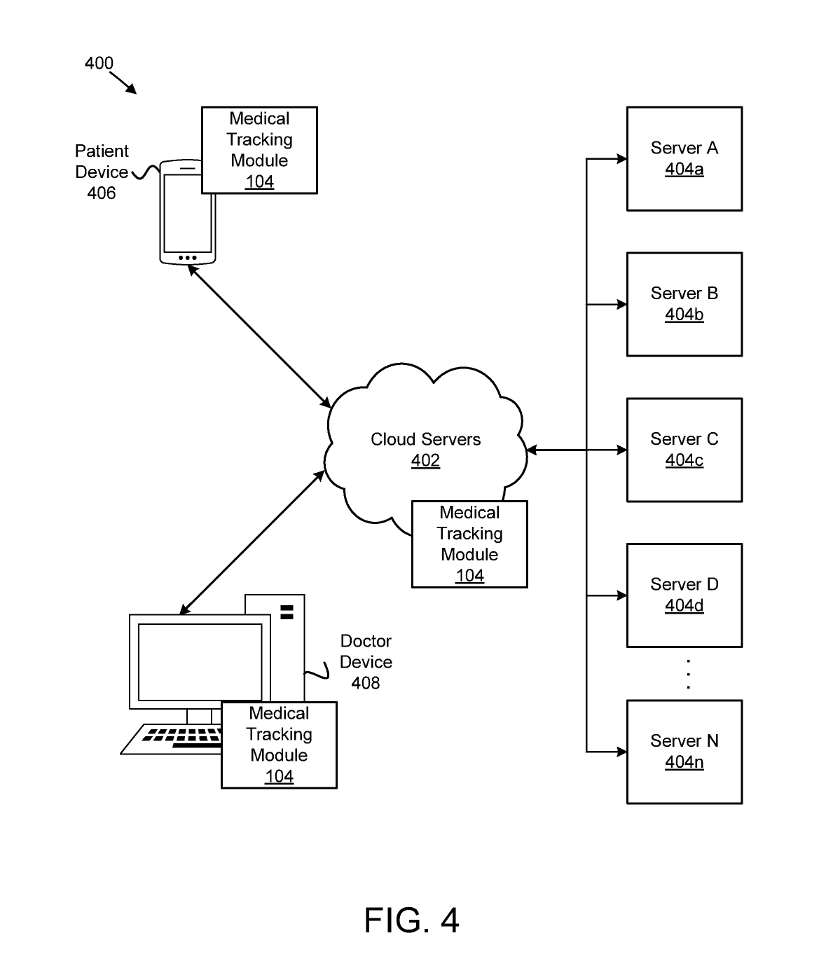

FIG. 4 depicts one embodiment of a system 400 for medical condition tracking and analysis. In one embodiment, the system 400 includes one or more cloud servers 402, a plurality of external data sources 404a-n, patient devices 406, doctor devices 408, and medical tracking modules 104. In one embodiment, a patient reports subjective and/or objective data, e.g., “interactive” objective data, using the patient device 406, such as a smart phone, tablet computer, laptop computer, or the like. The patient, for example, may log an event related to the medical condition, fill out a questionnaire or a survey, or the like. The data module 202 may receive the subjective and/or objective data and store the data on a cloud server 402. The cloud servers 402 may comprise dedicated servers, virtual servers, or the like, that are configured to store data and perform one or more statistical analyses on the stored data.

In certain embodiments, based on the patient's medical condition, the data module 202 receives one or more objective data sets, e.g., environmental data sets, from one or more external data sources 404a-n. For example, relevant objective data for an asthma patient may include air quality data, pollen data, pollution data, or the like for a particular location. The external data sources 404a-n may be associated with or maintained by one or more agencies. For example, Server A 404a may be a database of air quality data maintained by the Environmental Protection Agency. The data module 202 may associate the received objective data with a timestamp and store the objective data on a cloud server 402 along with the subjective and/or objective data provided by the patient.

In some embodiments, the trend module 204 determines a trend based on the subjective and objective data over a period of time, which indicates whether the patient's medical condition is trending in a positive direction, a negative direction, or is substantially constant. Based on the trend data determined by the trend module 204, the factor module 206 determines one or more factors that may affect the patient's medical condition, such as air quality factors, pollen levels, pollution data, or the like, for one or more data points of the trend data. In some embodiments, the factor module 306 determines one or more weights for the factors to indicate which factors may have a greater effect on the patient's medical condition than others.

In certain embodiments, the report module 306 sends a report of the trend data, the one or more factors, or the like information related the patient's medical condition from the cloud servers 402 to a device 408 associated with the patient's doctor. Moreover, the report module 306 may send reports comprising predicted, estimated, forecasted, or the like factors that may affect the patient's medical condition at some point in the future. In such an embodiment, the prediction module 304 determines one or more predicted factors that may have an effect on the patient's medical condition at a future period of time. In this manner, the doctor and/or the patient may proactively take steps to anticipate changes in the patient's medical condition due to possible changes in environmental or other factors.

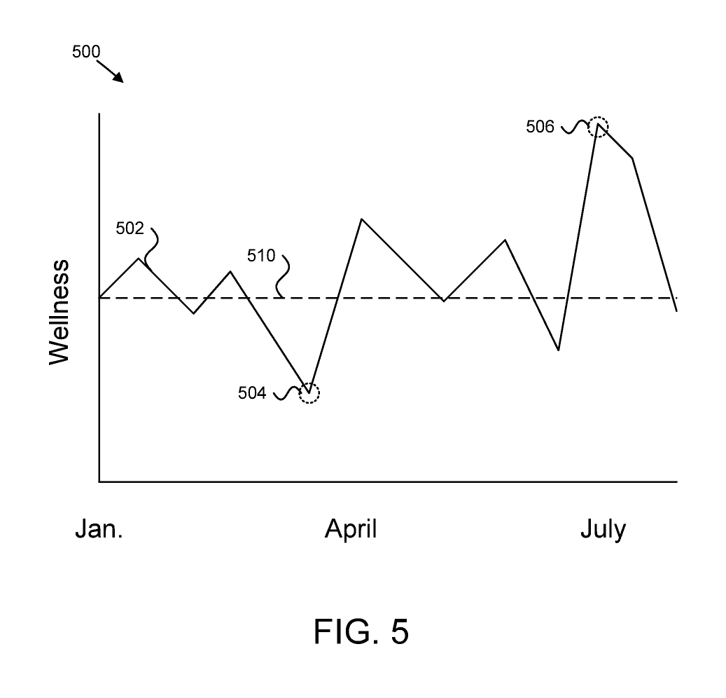

FIG. 5 depicts one embodiment of a trend graph 500 that depicts a trend line 502 for a patient. In the depicted embodiment, the trend line 502 is measured over a period of time, from January to July. The trend line 502 may be presented by the trend module 202 and includes various data points 504, 506 that represent how well the patient feels at a particular period of time, e.g., the patient's wellness status. In certain embodiments, the graph 500 includes a mid-line 510 that describes the average wellness status of a patient that has the medical condition at issue. In one embodiment, a patient that is trending below the mid-line 510 may be maintaining or controlling their medical condition poorly, while a patient that is trending above the mid-line 510 may be doing a good job at maintaining or controlling their medical condition.

In certain embodiments, the factor module 206 may analyze one or more data points 504, 506 of the trend data to determine one or more factors that contribute to the patient's medical condition. For example, in the depicted embodiment, the factor module 206 may select the outlier data points 504, 506 to determine one or more factors, based on the subjective and objective data (e.g., “interactive” objective data and/or environmental data), that may have affected the patient's wellness status for that time period. The factor module 206 may perform one or more statistical analyses, such a regression analyses, correlations, or the like, to determine important factors related to the patient's medical condition. Moreover, the factor module 206 may assign one or more calculated weights to the determined factors such that doctors and/or patients may be able to determine which factors have a greater effect on the patient's medical condition.

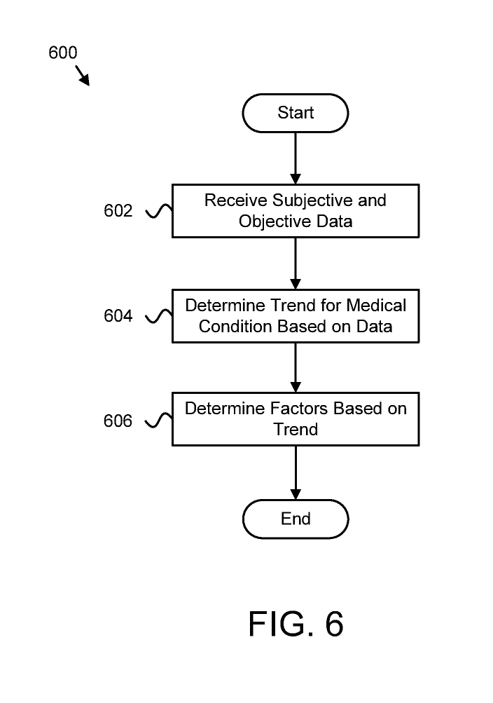

FIG. 6 depicts one embodiment of a method 600 for medical condition tracking and analysis. In one embodiment, the method 600 begins and the data module 202 receives 602 subjective data and objective data related to a medical condition. In a further embodiment, the trend module 204 determines 604 a trend for the medical condition based on the subjective and/or the objective data for a predetermined period of time. The factor module 206, in a further embodiment, determines 606 one or more factors that may affect the patient's medical condition based on the trend data, and the method 600 ends.

FIG. 7 depicts one embodiment of a method 700 for medical condition tracking and analysis. In one embodiment, the method 700 begins and the data module 202 receives 702 subjective data from a patient. In certain embodiments, the subjective data may be received in response to a patient filling out a questionnaire, survey, poll, quiz, or the like related to the patient's medical condition. In some embodiments, the subjective data may be received in response to a patient logging event data, such as a qualitative rating regarding how the patient feels at a particular moment.

In certain embodiments, the data module 202 receives 704 objective data from one or more external sources. In certain embodiments, the objective data comprises “interactive” subjective data inputted by a patient, e.g., the frequency with which the patient uses his inhaler in a day, the types of medications used by the patient and the frequency with which the medications are taken, and/or the like. In some embodiments, the objective data may comprise environment data, such as air quality data, pollen data, pollution data, or the like for a particular area. The external sources may comprise servers, databases, or the like that are maintained by third-party entities, organizations, or agencies, such as the EPA, the CDC, the NOAA, or the like. In a further embodiment, the trend module 204 performs 706 one or more statistical analyses on the subjective and objective data in order to determine a wellness status, score, rating, rank, or the like that describes the patient's medical condition.

The trend module 204, in a further embodiment, determines 708 a trend, adds to an existing trend, updates an existing trend, or the like of the patient's medical condition history. For example, the trend module 204 may add a data point based on the patient's self-reported subjective data that describes how the patient is feeling at a particular period of time to an existing set of trend data that describes the patient's medical condition over a period of time. The trend module 204 may add a data point to the trend that represents the wellness status for the patient.

The factor module 206 determines 710 one or more factors that may affect the patient's medical condition based on one or more data points of the trend data. For example, the factor module 206 may select a plurality of data points of the trend data where the patient's medical condition was in decline and perform one or more statistical analyses on the trend data (e.g., data comprising subjective and objective data sets), such as regression analyses or correlations, to determine one or more factors related to the medical condition. In such an embodiment, the factor module 206 may assign weights to the one or more factors that describe the level of impact that the factor may have on the patient's medical condition. For example, the factor module 20 may determine that certain pollen types have a greater effect on a patient that suffers from allergies than the time of day or the patient's age.

In one embodiment, the recommendation module 302 generates 712 one or more recommendations for controlling the patient's medical condition based on the determined factors. For example, the recommendation module 302 may recommend that an asthmatic patient avoid locations that contain high levels of pollen that the asthmatic patient may be susceptible to in response to the factor module 206 determining 710 that the particular pollen type exacerbates the patient's medical condition. In a further embodiment, the prediction module 304 forecasts, predicts, or otherwise estimates future objective data values and generates 712 predictions regarding the patient's medical condition based on the determined factors. For example, the prediction module 304 may forecast that the patient may suffer from allergic reactions within the next two weeks based on a history of a particular pollen, which has been determined to be a factor in the patient's medical condition, becoming widespread during that time period.

The report module 306, in certain embodiments, generates one or more reports based on the subjective and objective data, the trend data, one or more factors, or the like. The report module 306, in some embodiments, sends 714 a report of the results to a doctor for the patient and/or to the patient. In one embodiment, the report module 306 presents 716 the results of the report on an interface for a client device associated with the doctor. In this manner, both the patient and the doctor are kept up-to-date on the patient's current medical condition and can anticipate possible environmental changes, include preventive measures in the patient's treatment, and work together to help the patient maintain and control his or her medical condition, and the method 700 ends.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus for tracking a medical condition, the apparatus comprising:
a data module that receives subjective and objective data associated with a medical condition for a patient;
a trend module that determines a trend for the medical condition associated with the patient over a period of time based on the subjective and objective data; and
a factor module that identifies one or more factors that affect the medical condition, wherein one or more weighted values are calculated for the one or more factors based on the trend;
wherein at least a portion of the data module, the trend module, and the factor module comprise one or more of hardware and executable code, the executable code stored on one or more computer readable storage media.

2. The apparatus of claim 1, wherein the trend module updates the trend for the medical condition associated with the patient in real-time in response to receiving one or more of subjective and objective data.

3. The apparatus of claim 1, wherein the factor module determines the one or more weighted values by correlating one or more data points of the subjective data with one or more data points of the objective data.

4. The apparatus of claim 1, further comprising a recommendation module that generates one or more recommendations related to the medical condition based on the weighted values of the one or more factors.

5. The apparatus of claim 1, further comprising a prediction module that forecasts a state of the medical condition at a future point in time based on the one or more factors and forecasted objective data.

6. The apparatus of claim 1, further comprising a report module that generates one or more reports for the patient based on a state of the patient's medical condition, the one or more reports being accessible to a client device associated with the patient's doctor.

7. The apparatus of claim 1, further comprising a messaging module that generates one or more messages for a patient, the one or more messages being sent in response to input received from the patient's doctor.

8. The apparatus of claim 1, wherein the factor module identifies the one or more factors based on one or more outlier data points of the determined trend, the outlier data points indicating one or more abnormalities associated with a state of the patient's medical condition.

9. The apparatus of claim 1, wherein the subjective data comprises qualitative feedback provided by the patient and related to the medical condition, the patient feedback being received from a client device associated with the patient.

10. The apparatus of claim 1, wherein the subjective data comprises event data received from a client device associated with the patient, the event data comprising a self-reported measurement of the medical condition associated with the patient at a point in time.

11. The apparatus of claim 1, wherein the objective data comprises environmental data, the environmental data being received from one or more remote servers storing the environmental data.

12. The apparatus of claim 11, wherein the environmental data comprises one or more of weather data, air quality data, pollen data, pollution data, ozone data, pressure data, and particulate matter data.

13. The apparatus of claim 1, wherein the objective data comprises data reported by the patient, the data comprising one or more quantifiable factors associated with the medical condition.

14. A system for tracking a medical condition, the system comprising:
a client device associated with a patient;
a client device associated with a doctor for the patient; and
a remote server communicatively coupled to the client device associated with a patient and the client device associated with the doctor for the patient, the remote server comprising:
a data module that receives subjective and objective data associated with a medical condition for a patient;
a trend module that determines a trend for the medical condition associated with the patient over a period of time based on the subjective and objective data; and
a factor module that identifies one or more factors that affect the medical condition, wherein one or more weighted values are calculated for the one or more factors based on the trend;
wherein at least a portion of the data module, the trend module, and the factor module comprise one or more of hardware and executable code, the executable code stored on one or more computer readable storage media of the remote server.

15. The system of claim 14, wherein the trend module performs one or more statistical analyses on the subjective and objective data on the remote server, the trend being generated as a function of one or more of the statistical analyses.

16. The system of claim 15, further comprising a report module that sends the results of the one or more statistical analyses to the client device associated with the doctor of the patient.

17. The system of claim 16, wherein the report module presents the results of the one or more statistical analyses on a graphical interface for the client device associated with the doctor of the patient.

18. The system of claim 14, wherein the objective data is received from one or more external data sources comprising one or more remote servers storing environmental data, the environmental data associated with the patient's medical condition.

19. A method for tracking a medical condition, the method comprising:
receiving, by a processor, subjective and objective data associated with a medical condition for a patient;
determining a trend for the medical condition associated with the patient over a period of time based on the subjective and objective data; and
identifying one or more factors that affect the medical condition, wherein one or more weighted values are calculated for the one or more factors based on the trend.

20. The method of claim 19, further comprising forecasting a state of the medical condition at a future point in time based on the one or more factors and forecasted objective data.
